(19) United States
(12) Patent Application Publication
(10) Pub. No.: US 2024/0285551 A1
(43) Pub. Date: Aug. 29, 2024

(54) A SMALL MOLECULE TNF-a INHIBITOR AND PREPARATION METHOD AND USE THEREOF

(71) Applicant: FUDAN UNIVERSITY, Shanghai (CN)

(72) Inventors: Xun SUN, Shanghai (CN); Meilin TANG, Shanghai (CN); Xiaoyan SHEN, Shanghai (CN)

(21) Appl. No.: 18/571,013

(22) PCT Filed: Jun. 6, 2022
(86) PCT No.: PCT/CN2022/097060

§ 371 Date: Dec. 15, 2023

(30) Foreign Application Priority Data
Jun. 16, 2021 (CN) 202110664043.X

(51) Int. Cl. A61K 31/122; A61P 37/06; C07C 45/65; C07C 49/755; C07C 315/04

(52) U.S. Cl. CPC A61K 31/122; A61P 37/06; C07C 45/65; C07C 49/755; C07C 315/04; C07C 2602/08

(57) ABSTRACT

The present invention provides a small molecule TNF-α inhibitor STU104 and a preparation method and use thereof, wherein an absolute configuration of an optical isomer (R)-STU104 is identified, especially it has found use of STU104 for treatment of autoimmune inflammatory diseases mediated due to TNF-α overexpression including ulcerative colitis, Crohn's disease, rheumatoid arthritis, osteoarthritis, alopecia areata, Sjogren's syndrome, lupus erythematosus, dermatomyositis, etc. The present invention has broad clinical therapeutic significance.

### TECHNICAL FIELD

The present invention relates to the field of pharmaceutical chemistry technology, specifically to a small molecule TNF-α inhibitor and a preparation method and use thereof.

### BACKGROUND ART

Ulcerative colitis (UC) as a non-specific autoimmune inflammatory bowel disease is known together with Crohn's disease (CD) as Inflammatory Bowel Disease (IBD). It is clinically manifested by acute and recurrent bloody mucinous diarrhea, with a cure period up to several months and years, and even lifelong inability to cure, and even malignant transformation into colon cancer due to DNA damage and microsatellite instability of mucosal cells resulting from repeated disease attacks. With the improvement of people's living standard and the change of diet structure, the incidence rates of UC and its related colon cancer are on the rapid rise, with faster growth rates in cities and higher incidence rates in the younger population. Ulcerative colitis is the leading cause of malignant colon cancer, with a malignant transformation rate of up to 40%. The prevalence rate of colon cancer in China has reached 13%, with more than 172,000 patients per year, and the mortality rate thereof is 8.0%, ranking sixth and fifth respectively in the incidence and mortality rates of malignant tumors; the mortality rate of colon cancer is higher globally, reaching around 9.0%, ranking fourth among malignant tumors. As can be seen, the high incidence of UC and colon cancer has become a major global health issue that urgently needs to be addressed.

Due to the unclear pathogenesis of UC or IBD, there is yet no effective method to cure UC or IBD. At present, traditional small molecule therapeutic drugs such as 5-aminosalicylic acid (5-ASA), corticosteroid prednisone or dexamethasone, as well as immunosuppressants represented by imidazole mercaptopurine or the like are mostly used in clinical practice. Although they can temporarily alleviate and mitigate the inflammatory symptoms of patients to varying degrees, they cannot completely or effectively control the onset or development of the disease; and patients need to take medication for life and are prone to serious infection, thrombosis, and increased risks of malignant tumors and other adverse reactions. In addition, a JAK inhibitor tofacitinib which is a small molecule targeted drug has been approved for the treatment of ulcerative colitis in the year of 2018, but this type of inhibitor easily causes pulmonary embolism and thus has received black box warnings from the FDA. A biological macromolecular immunosuppressant vedolizumab as an α4β7-integrin heterodimer antagonist is currently the most effective biological drug for treating UC worldwide, but it easily causes upper respiratory tract infections, nasopharyngeal inflammation, headache, arthritis, nausea, fever, and other adverse reactions, and its high cost prohibits its widespread use. Therefore, UC has been listed as a major refractory inflammatory and immune chronic disease in the global medical field, and has become a hot topic in the research and development of the current pharmaceutical industry and an urgent clinical problem to be solved.

In recent years, with the continuous deepening of research, a large number of medical studies have proven that the occurrence and development of UC are closely related to the generation, i.e., excessive secretion of an inflammatory factor TNF-α in an organism, that is, the excessive secretion of TNF-α may mediate the expression of inflammatory genes in the pro-inflammatory cell signaling pathway by attacking normal intestinal epithelial cells in the intestine, and may further facilitate the synthesis of various inflammatory cytokines. Therefore, the effective inhibition of generation of TNF-α or interference with the functional effects of upstream and downstream targets (TLR4, MYD88, JNK, ERK, P38, TAK1, MKK3, NIK, TACE, etc.) in the signaling pathway can be used as a research and development strategy for anti-UC therapeutic drugs. However, at present, small molecule inhibitors for inhibiting generation of TNF-α for anti-UC clinical indications are not yet commercially available, and thus preparation of small molecule inhibitors against the effective target TNF-α as medicaments for treating UC is an urgent issue to be solved in the art.

### SUMMARY OF THE INVENTION

To overcome the shortcomings in the prior art, the present invention is implemented through the following technical solutions.

In a first aspect, the present invention provides a small molecule TNF-α inhibitor or a pharmaceutically acceptable salt thereof, wherein its structure is represented by formula I:

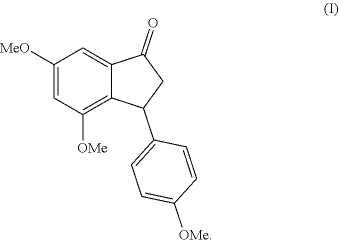 [SMILES: COc1ccc(C2CC(=O)c3cc(OC)cc(OC)c32)cc1]

Further, said small molecule TNF-α inhibitor is racemic (±)-STU104 or (R)-STU104 (i.e., 4,6-dimethoxy-3R-(4-methoxyphenyl)-2,3-dihydro-1H-indanone) with a structure represented by formula II or III:

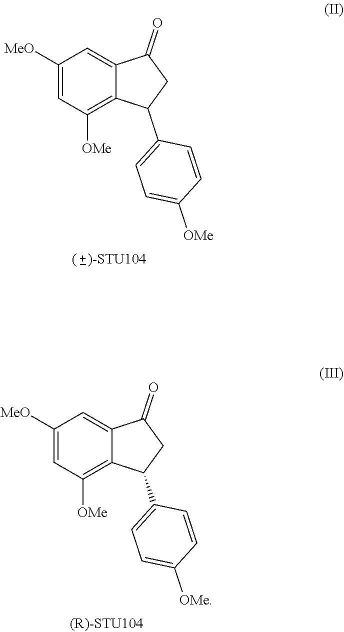

In a second aspect, the present invention provides a method for preparation of said small molecule TNF-α inhibitor (R)-STU104 of formula III, which is prepared according to the following route:

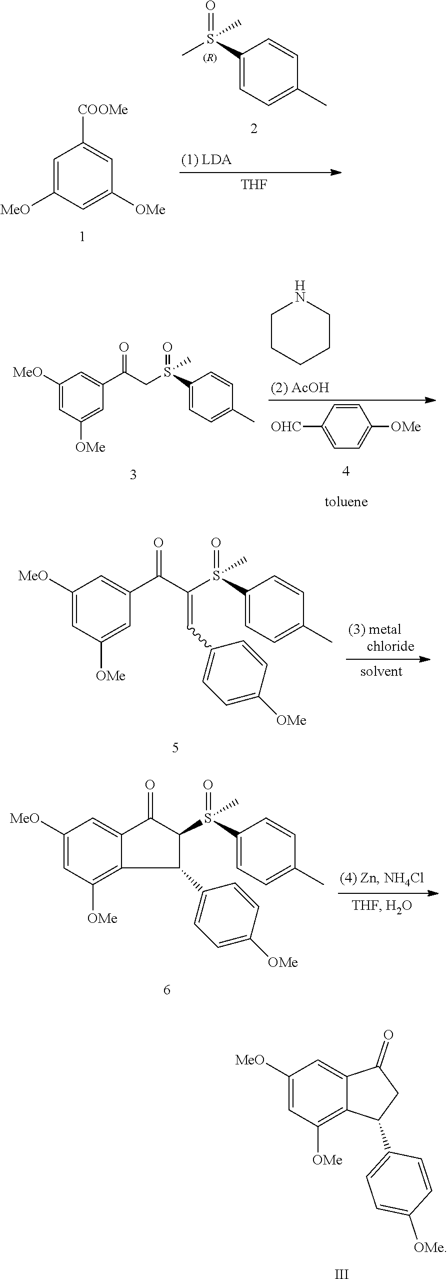

Further, in Step (1), a synthetic reaction of Compound 3 from Compounds 1 and 2 is controlled at a temperature of −80 to −60° C.

Further, in Step (2), a synthetic reaction of Compound 5 from Compounds 3 and 4 is controlled at a temperature of 90 to 120° C.

Further, in Step (3), a synthetic reaction of Compound 6 from Compound 5 is controlled at a temperature of 30 to 50° C.

Further, in Step (3), a metal chloride used in the synthetic reaction of Compound 6 from Compound 5 is AlCl₃ or ZnCl₂.

Further, in Step (3), a solvent used in the synthetic reaction of Compound 6 from Compound 5 is toluene, dichloromethane, or 1,2-dichloroethane.

In a third aspect, the present invention provides use of said small molecule TNF-α inhibitor in preparation of medicaments for treating autoimmune inflammatory diseases.

Further, said autoimmune inflammatory diseases are caused by overexpression of TNF-α or overexpression of mRNAs thereof.

Further, said use is to have an effect of inhibiting or reducing TNF-α release. Furthermore, said use is to reduce a phosphorylation level of MKK3 by regulating TAK1 within inflammatory cells, and further mediate inhibition or reduction of phosphorylation levels of proteins including but not limited to p38, MnK1, MK2, and/or elF4E in a downstream signaling pathway of MKK3 to achieve the effect of inhibiting or reducing the TNF-α release.

Further, said autoimmune inflammatory diseases include but are not limited to ulcerative bowel diseases, rheumatoid arthritis, osteoarthritis, alopecia areata, Sjogren's syndrome, lupus erythematosus, dermatomyositis, etc.

Furthermore, said ulcerative bowel diseases include but are not limited to acute ulcerative colitis, chronic ulcerative colitis, or Crohn's disease.

Furthermore, treatment of said acute ulcerative colitis or said chronic ulcerative colitis is to have an effect of inhibiting overexpression of at least one of optional inflammatory factors TNF-α, IL-1β, IL-6, and IL-23 or overexpression of mRNAs thereof.

Furthermore, said treatment of said chronic ulcerative colitis is dose-dependent.

Furthermore, treatment of said acute ulcerative colitis, said chronic ulcerative colitis, or said Crohn's disease includes but is not limited to improvement in symptoms and reduction in mortality rates. Further, said improvement in symptoms includes but is not limited to inhibiting weight loss, reducing hematochezia, and inhibiting colon shortening, ulcers, scars, and/or edema.

### Beneficial Effects

The present invention has been experimentally proven to have the following beneficial effects.

1. The racemic (±)-STU104 or optical isomer (R)-STU104 as claimed in the present invention has specific inhibitory effects in vitro on overexpression of the inflammatory factor TNF-α produced by lipopolysaccharide (LPS) induced macrophages (RAW264.7) and overexpression of mRNAs thereof, with (R)-STU104 being superior to (S)-STU104 in activity.

2. The racemic (±)-STU104 or optical isomer (R)-STU104 as claimed in the present invention has specific inhibitory effects on overexpression of such inflammatory factors as TNF-α, IL-1β, IL-6, and IL-23 in the serum of mice with ulcerative colitis and overexpression of mRNAs thereof, thus being superior in activity to its optical isomer (S)-STU104.

3. The racemic (±)-STU104 or optical isomer (R)-STU104 as claimed in the present invention has superior pharmacological effects on ulcerative bowel diseases (including acute ulcerative colitis, chronic ulcerative colitis, and Crohn's disease) in vivo compared to a clinical positive control drug mesalazine, thus being significantly superior to the optical isomer (S)-STU104.

4. The present invention further provides a method for asymmetric synthesis of the compound (R)-STU104, thus greatly facilitating its industrial application.

### BRIEF DESCRIPTION OF THE DRAWINGS

In FIG. 1, A1 and B1 illustrate histograms for inhibitory effects of (R)-STU104 and (±)-STU104 on release of the inflammatory factor TNF-α by lipopolysaccharide (LPS) induced macrophages (RAW264.7), respectively; A2 and B2 illustrate graphs for inhibitory effects of (R)-STU104 and (±)-STU104 on release of the inflammatory factor TNF-α by lipopolysaccharide (LPS) induced macrophages (RAW264.7), respectively.

In FIG. 2, A illustrates western blots of phosphorylated proteins related to signaling pathways where release of the inflammatory factor TNF-α by lipopolysaccharide (LPS) induced macrophages (RAW264.7) is inhibited by (R)-STU104 in a concentration dependent manner; and B illustrates relative contents of phosphorylated proteins related to signaling pathways where release of the inflammatory factor TNF-α is inhibited by (R)-STU104 at low, medium, and high doses, as compared to a p38 inhibitor positive control, LPS group, and blank control group (“*” represents p<0.05; “**” represents p<0.01; and “***” represents p<0.001).

FIG. 3 illustrates respective therapeutic recovery effects of (R)-STU104, (S)-STU104, (±)-STU104, blank control group, model group, and Mes (mesalazine, positive control) on a DSS induced acute ulcerative colitis model in C57 mice (hereinafter referred to as “acute UC mice”; “*” represents p<0.05; “**” represents p<0.01; and “***” represents p<0.001), wherein A1 is a graph of weight changes for each group of acute UC mice within 7 days of administration; A2 is a histogram of body weight recovery rates for each group of acute UC mice after 7 days of administration; B1 is a statistical chart of colitis pathological scores for each group of acute UC mice within 7 days; B2 is a histogram of colitis recovery rates for each group of acute UC mice within 7 days; C1 shows colon length changes for each group of acute UC mice after 7 days of administration; C2 is a histogram of colon length recovery rates for each group of acute UC mice after 7 days of administration; D1 shows colon pathological sections of the acute ulcerative colitis for each group of acute UC mice; D2 shows comparison of scores for colon pathological sections of the acute ulcerative colitis for each group of acute UC mice; and D3 shows recovery rates of colon pathological sections for each group of acute UC mice.

FIG. 4 illustrates inhibitory effects of (R)-STU104, (S)-STU104, (±)-STU104, and positive control Mes group (mesalazine) on inflammatory factors as treatment endpoints of the DSS induced acute ulcerative colitis model in C57 mice, wherein A1, B1, C1, and D1 represent respective effects on protein contents of such inflammatory factors as TNF-α, IL-1β, IL-6, and IL-23 in the serum of acute UC mice; A2, B2, C2, and D2 represent respective inhibition ratios of protein contents of such inflammatory factors as TNF-α, IL-1β, IL-6, and IL-23 in the serum of acute UC mice; E1, F1, G1, and H1 represent respective effects on mRNA contents of such inflammatory factors as TNF-α, IL-1β, IL-6, and IL-23 in colon tissues of acute UC mice; and E2, F2, G2, and H2 represent respective inhibition ratios of mRNA contents of such inflammatory factors as TNF-α, IL-1β, IL-6, and IL-23 in colon tissues of acute UC mice.

FIG. 5 illustrates therapeutic recovery effects of (R)-STU104 on the chronic ulcerative colitis model of IL-10 whole gene knockout mice (hereinafter referred to as “chronic UC mice”) at low, medium, and high doses as compared to the blank control group, model group, and Mes (mesalazine) positive control group (“*” represents p<0.05; “**” represents p<0.01; and “***” represents p<0.001), wherein A1, A2, B1, B2, C1, C2, D1, D2, and D3 represent effects of the compound (R)-STU104 on the changes in survival rate, comparison of survival rates, weight changes, comparison of body weight recoveries, changes in colon length, comparison of colon length recovery rates, effects on colon pathological sections, comparison of scores for colon pathological sections, and recovery rates of colon pathological sections in the chronic UC mice, respectively.

FIG. 6 illustrates inhibitory effects of (R)-STU104 on inflammatory factors as model experiment endpoints for the IL-10 whole gene knockout mice at low, medium, and high doses as compared to the blank control group, model group, and Mes (mesalazine) positive control group, wherein A1, B1, C1, and D1 represent respective effects of the compound (R)-STU104 on protein contents of such inflammatory factors as TNF-α, IL-1β, IL-6, and IL-23 in the serum of chronic UC mice; A2, B2, C2, and D2 represent inhibition ratios of protein contents of such inflammatory factors as TNF-α, IL-1β, IL-6, and IL-23 in the serum of chronic UC mice by the compound (R)-STU104; E1, F1, G1, and H1 represent effects of the compound (R)-STU104 on mRNA contents of such inflammatory factors as TNF-α, IL-1β, IL-6, and IL-23 in colon tissues of chronic UC mice; and E2, F2, G2, and H2 represent inhibition ratios of mRNA contents of such inflammatory factors as TNF-α, IL-1β, IL-6, and IL-23 in colon tissues of chronic UC mice by the compound (R)-STU104.

### DETAILED DESCRIPTION OF THE EMBODIMENTS

A further detailed description of the present invention will be provided below in conjunction with the accompanying drawings and specific embodiments.

### Example 1: Synthesis and Identification of Compound (±)-STU104 (II)

The synthesis of compound (±)-STU104 (II) can be found in a Chinese patent CN107082743B with structural identification data as follows: compound (±)-STU104 (II) is a white solid (at a yield of 98%); molecular weight: 298; optical rotation value: [α]_{D}²⁰=0 (c=0.14, CHCl₃); ¹H-NMR (400 Hz, CDCl₃) δ 6.97 (d, J=8.0 Hz, 2H), 6.84 (d, J=2.0 Hz, 1H), 6.78 (d, J=8.0 Hz, 2H), 6.63 (d, J=2.0 Hz, 1H), 4.53 (dd, J=2.4 Hz, J=8.0 Hz, 1H), 3.85 (s, 3H), 3.77 (s, 3H), 3.66 (s, 3H), 3.19 (dd, J=8.0 Hz, J=19.2 Hz 1H), 2.58 (dd, J=2.4 Hz, J=19.2 Hz 1H); ¹³C-NMR (150 MHz, CDCl₃) δ 205.94, 161.00, 157.40, 157.21, 139.15, 138.33, 135.40, 127.35, 113.15, 105.52, 95.15, 55.14, 54.97, 54.59, 47.14, 39.85. HRMS (ESI) Calcd for C₁₈H₁₈O₄ (M+H): 299.1283; Found: 299.1280.

### Example 2: Synthesis and Identification of Compound (R)-STU104 (Formula III)

The compound of formula III, i.e., (R)-STU104 is obtained through a preparation method following the following route:

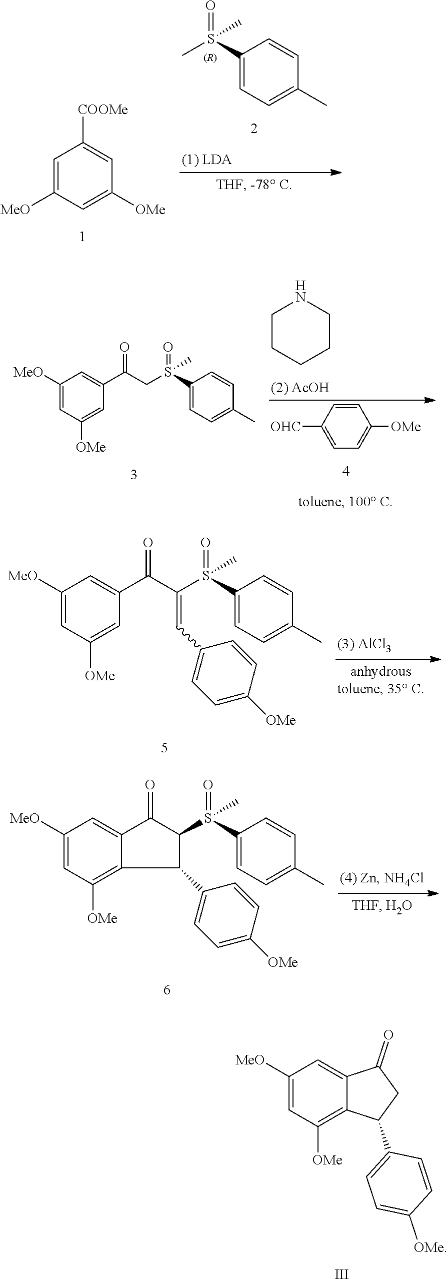

Step 1: (1) Synthesis of compound 3: a two-necked flask was taken and vacuumed under N₂ protection; (R)-methyl-p-methylphenyl sulfoxide (2) was first added and dissolved under stirring with anhydrous THF at −78° C.; lithium diisopropylamide (LDA) (1.2 eq) was slowly added to react for 1 hour; compound 1 (1.5 eq) was then added and stirred overnight; and a TLC test was conducted (with a volume ratio of PE:EA in the developing agent system being 2:1) to observe whether the raw material reacted completely. After the reaction was completed, it was concentrated under reduced pressure, extracted, and then washed sequentially with saturated NH₄Cl, water, and saturated NaCl solutions. The organic layer was dried over anhydrous Na₂SO₄, concentrated, and purified on a silica gel column (with a volume ratio of PE:EA in the eluent system being 2:1 to 1:1) to obtain the target compound 3 as a white powder.

Step 2: Synthesis of target compound (R)-STU104 (Formula III):

(2) Synthesis of compound 5: a two-necked flask was taken and vacuumed under N₂ protection; compound 3 and aldehyde 4 (1.2 eq) were added and dissolved under stirring with anhydrous toluene (Toluene), followed by addition of piperidine (0.2 eq) and acetic acid (0.2 eq) and stirring under reflux at 110° C. for 6 to 8 hours; and a TLC test was conducted (PE:EA=3:1) to observe whether the raw material reacted completely. After the reaction was completed, it was concentrated under reduced pressure, extracted with ethyl acetate (EA), and then washed sequentially with saturated NH₄Cl, water, and saturated NaCl solutions. The organic layer was dried over anhydrous Na₂SO₄, concentrated, and purified on a silica gel column (with a volume ratio of PE:EA in the eluent system being 3:1 to 2:1) to obtain the target compound 5 as a light yellow oil.

(3) Synthesis of compound 6: a two-necked flask was taken and vacuumed under N₂ protection; compound 5 was added and dissolved under stirring with anhydrous toluene; AlCl₃ (1.1 eq) was added and reacted at 35° C., followed by stirring for 12 to 24 hours; and a TLC test was conducted (with a volume ratio of PE:EA in the developing agent system being 3:1) to observe whether the raw material reacted completely. After the reaction was completed, it was concentrated under reduced pressure, extracted with EA, and then washed sequentially with saturated NH₄Cl, water, and saturated NaCl solutions. The organic layer was dried over anhydrous Na₂SO₄ and concentrated to obtain the compound 6 which was directly put into the next reaction step without separation.

(4) Synthesis of target compound (R)-STU104: a two-necked flask was taken and vacuumed under N₂ protection; compound 6 was added and dissolved under stirring with THF; a saturated ammonium chloride solution and a Zn powder (1.2 eq) were added and stirred at room temperature for 1 hour; and a TLC test was conducted (with a volume ratio of PE:EA in the eluent system being 5:1) to observe whether the raw material reacted completely. After the reaction was completed, it was concentrated under reduced pressure, extracted with EA, and then washed sequentially with saturated NH₄Cl, water, and saturated NaCl solutions. The organic layer was dried over anhydrous Na₂SO₄, concentrated, and purified on a silica gel column (with a volume ratio of PE:EA in the eluent system being 8:1 to 5:1) to obtain the target compound (R)-STU104 as a white powder.

The physicochemical identification data of intermediates and products in each step are as below.

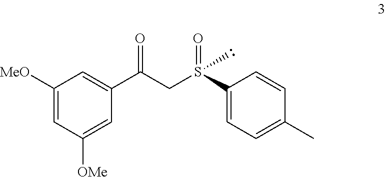

Compound 3 is a white powder (at a yield of 95%). ¹H-NMR (400 MHZ, CDCl₃) δ 7.58 (d, J=8.0 Hz, 2H), 7.31 (d, J=8.0 Hz, 2H), 7.00 (d, J=4.0 Hz, 2H), 6.66 (t, J=4.0 Hz, 1H), 4.52 (d, J=12 Hz, 1H), 4.23 (d, J=12 Hz, 1H), 3.81 (s, 6H), 2.40 (s, 3H); ¹³C-NMR (150 MHz, CDCl₃) δ 190.44, 160.32, 141.62, 139.45, 137.27, 129.42, 123.72, 105.90, 65.72, 55.03, 20.84. HRMS (ESI) Calcd for C₁₇H₁₈O₄S (M+H): 319.1004; Found: 319.1002.

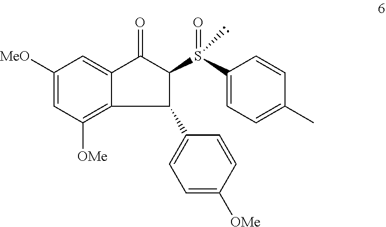

Compound 6 is a white solid. ¹H-NMR (400 MHZ, CDCl₃) δ 7.53 (d, J=8.0 Hz, 2H), 7.33 (d, J=8.0 Hz, 2H), 6.85 (d, J=2.0 Hz, 1H), 6.64 (d, J=2.0 Hz, 1H), 6.52 (d, J=8.0 Hz, 2H), 6.27 (d, J=8.0 Hz, 2H), 4.77 (d, J=2.0 Hz, 1H), 3.85 (s, 3H), 3.68 (s, 3H), 3.62 (s, 3H), 3.56 (d, J=2.0 Hz, 1H), 2.45 (s, 3H); ¹³C-NMR (150 MHz, CDCl₃) δ 198.54, 161.32, 157.36, 156.77, 141.07, 138.54, 137.97, 137.92, 133.20, 129.33, 127.31, 123.76, 112.94, 106.73, 95.49, 78.94, 55.19, 54.98, 54.48, 38.16, 20.81. HRMS (ESI) Calcd for C₂₅H₂₄O₅S (M+H): 437.1423; Found: 437.1420.

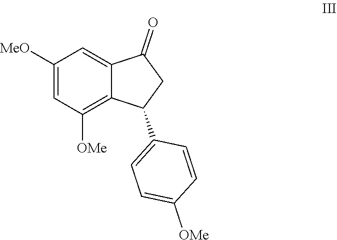 [SMILES: COc1ccc([C@H]2CC(=O)c3cc(OC)cc(OC)c32)cc1]

Compound (R)-STU104 (Formula III, 4,6-dimethoxy-3R-(4-methoxyphenyl)-2,3-dihydro-1H-indanone) is a white solid (at a yield of 98%); molecular weight: 298; optical rotation value: [α]_{D}²⁰=−36° (c=0.14, CHCl₃); ¹H-NMR (400 Hz, CDCl₃) δ 6.97 (d, J=8.0 Hz, 2H), 6.84 (d, J=2.0 Hz, 1H), 6.78 (d, J=8.0 Hz, 2H), 6.63 (d, J=2.0 Hz, 1H), 4.53 (dd, J=2.4 Hz, J=8.0 Hz, 1H), 3.85 (s, 3H), 3.77 (s, 3H), 3.66 (s, 3H), 3.19 (dd, J=8.0 Hz, J=19.2 Hz 1H), 2.58 (dd, J=2.4 Hz, J=19.2 Hz 1H); ¹³C-NMR (150 MHz, CDCl₃) δ 205.94, 161.00, 157.40, 157.21, 139.15, 138.33, 135.40, 127.35, 113.15, 105.52, 95.15, 55.14, 54.97, 54.59, 47.14, 39.85. HRMS (ESI) Calcd for C18H1804 (M+H): 299.1283; Found: 299.1280.

### Example 3: Pharmacological Activities of Compounds (±)-STU104 (II) and (R)-STU104 (III)

### 3.1 Effects of Compounds (±)-STU104 (II) and (R)-STU104 (III) on Release of the Inflammatory Factor TNF-α by Lipopolysaccharide (LPS) Induced Macrophages (RAW264.7).

As shown in FIG. 1, cells from the macrophage cell line RAW264.7 cultured in vitro were employed. LPS (1 μg/ml) and different concentrations of (±)-STU104 or (R)-STU104 (0.1 to 50 μM) were added respectively after the cells grew to a fused state, and incubated together at 37° C. under 5% CO₂ for 24 hours. A blank control group was established, and RT-PCR was employed to detect the inhibition states of TNF-α in cells by these compounds. The results showed that the IC₅₀ of (R)-STU104 with respect to the release of the inflammatory factor TNF-α by lipopolysaccharide (LPS) induced macrophages (RAW264.7) was 0.58 μM (see panel A2 in FIG. 1), and the IC₅₀ of the racemic STU104 with respect to the inflammatory factor TNF-α was 1.27 μM (see panel B2 in FIG. 1). As can be seen, the compound (±)-STU104 or (R)-STU104 has specific inhibitory effects in vitro on the mRNA overexpression level of the inflammatory factor TNF-α produced by LPS stimulated macrophages RAW264.7 and these effects are significantly superior to the compound (S)-STU104.

### 3.2 Inhibitory Mechanism of Compound (R)-STU104 on the Release of the Inflammatory Factor TNF-α.

To further clarify the inhibitory mechanism of (R)-STU104 on TNF-α release, we detected the effects of (R)-STU104 on phosphorylation levels of proteins related to the p38 signaling pathway (see FIG. 2). The p38 inhibitor (SB203580) was used as a positive control, and western blotting was used to detect protein expression levels. The results showed that (R)-STU104 was capable of inhibiting, in a concentration dependent manner, the phosphorylation of key proteins such as Mnk1, MK2, and elF4E in the p38 signaling pathway of RAW264.7 cells (3.125 μM, 6.25 μM, and 12.5 μM) induced by LPS (500 μg/L), ultimately leading to a decrease in the production of the target factor TNF-α; as compared to LPS, it had the same efficacy as the positive drug p38 inhibitor (SB203580, 12.5 μmol/L), with “*” representing p<0.05; “**” representing p<0.01; and “***” representing p<0.001. In addition, we tested the effect of (R)-STU104 on an upstream kinase TAK1 of MKK3, and the results showed no inhibition of the phosphorylation of TAK1. Therefore, the results showed that (R)-STU104 was capable of acting on MKK3 to block the interaction between proteins TAK1 and MKK3, thereby inhibiting MKK3 phosphorylation and further selectively inhibiting the phosphorylation of all proteins (p38→MnK1→MK2→elF4E) in the downstream signaling pathway of MKK3 in inflammatory cells of the colon, and, by lowering their phosphorylation levels, ultimately achieving the inhibition of release of the inflammatory factor TNF-α, finally reducing the target factor TNF-α, and achieving the anti-colitis effect.

### 3.3 Comparison of Compounds (R)-STU104, (S)-STU104, and (±)-STU104 in the Treatment of DSS Induced Acute Ulcerative Colitis in C57 Mice.

### 3.3.1 Grouping and Modeling

In order to verify the indications for the anti-ulcerative colitis activity of (R)-STU104, (S)-STU104, and (±)-STU104 in vivo, we constructed a DSS induced mouse inflammatory bowel disease model: thirty-six C57 male mice (aged 6-8 weeks, around 20 g) were taken and randomly divided into a normal control group (Control group), a DSS group (Model group), a positive control mesalazine group (50 mg/kg), a (R)-STU104 group (10 mg/kg), a (S)-STU104 group (10 mg/kg), and a (±)-STU104 group (10 mg/kg), with 10 mice in each group. Except for the normal group, all other groups of mice were allowed to freely drink a 3% DSS solution which was freshly prepared and replaced the next day (wherein the 3% DSS solution was prepared by weighing 3 g of a DSS powder (from MP Biomedicals, USA with a molecular weight of 36,000 to 50,000) and fully dissolving it in 100 mL of the drinking water for the mice); whereas the normal group was given blank drinking water; the control group mice drank water normally for 7 days, while the DSS group mice drank the 3% DSS solution freely for 7 days and were administrated for 6 days from the second day DSS was given. The mice were administered by gavage once a day with a volume of 10 mL/kg, wherein the normal control group and DSS group were gavaged with a solvent of 0.5% CMC-Na (wherein a 0.5% CMC-Na solution is at a concentration of 0.5 g/100 mL, the same below), while the other groups were separately gavaged with drugs dissolved in 0.5% CMC-Na.

### 3.3.2 Effects of Compounds (R)-STU104, (S)-STU104, and (±)-STU104 on Weight Changes of DSS Induced Acute UC Mice.

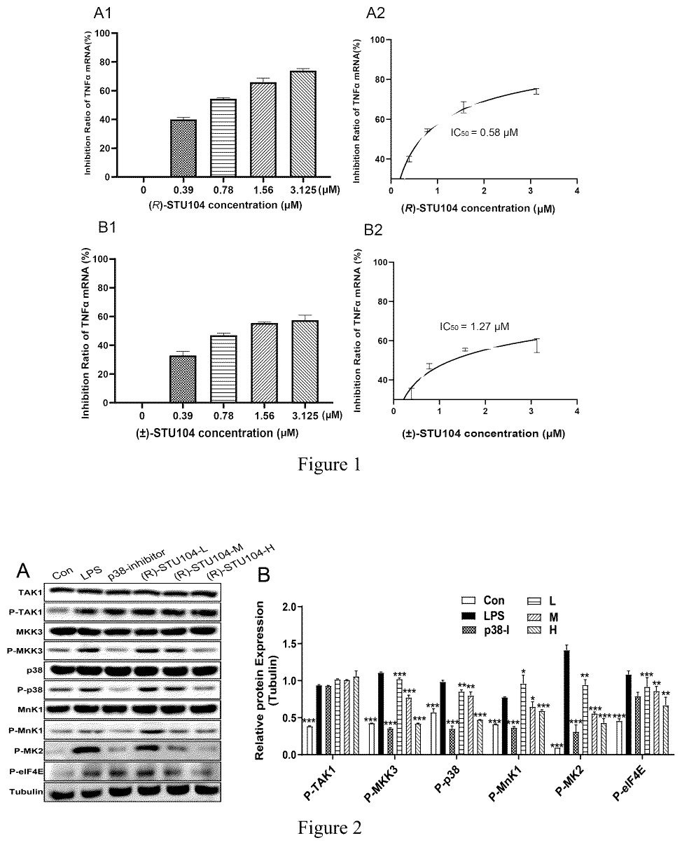

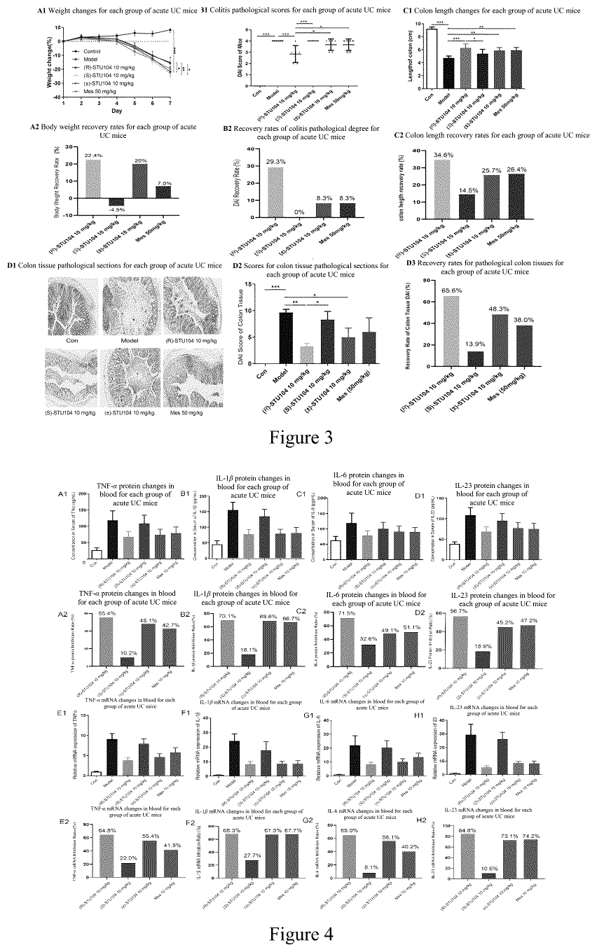

The normal control group mice were vivacious with glossy fur, stable weight gain, and normal stools appearing elliptical or spindle shaped. The DSS model group mice gradually began to show a decrease in autonomous activity, delayed response, decreased appetite, and metal lethargy, and the mice showed a decreasing trend in body weight throughout the experiment (see panel A1 in FIG. 3). On the 7th day of the experiment, the average weight of the DSS model group was significantly different, i.e., lower than that of the normal control group. The results showed that mice of the (R)-STU104 (10 mg/kg), (±)-STU104 (10 mg/kg), and positive drug mesalazine (50 mg/kg) groups showed significantly lighter symptoms and significantly slower trends in weight loss than those of the model group, with recovery rates of 22.4%, 20.0%, and 7%, respectively (see panel A2 in FIG. 3). The (R)-STU104 group (10 mg/kg) or (±)-STU104 group (10 mg/kg) was superior to the positive drug mesalazine group (50 mg/kg). On the contrary, the improvement of acute UC symptoms in mice of the (S)-STU104 group (10 mg/kg) was significantly lower than that in the mesalazine group (50 mg/kg).

### 3.3.3 Effects of Compounds (R)-STU104, (S)-STU104, and (±)-STU104 on Disease Activity Index (DAI) Scores in DSS Induced Acute UC Mice.

In order to further evaluate the disease development for each group of acute UC mice, we performed DAI scoring on the loose and bloody stools etc. for each mouse (see panel B1 in FIG. 3). Mice started to have loose stools on the 3^{rd} day of drinking DSS and showed obvious bloody, mucous, or mucopurulent bloody stools on the 5th day. As the experiment progressed, the disease became gradually worsened, with some animals experiencing severe bloody stools and significant emaciation. On the 7th day of the experiment, the DAI score of the DSS model group mice was significantly increased, while the DAI scores of the mice of the (R)-STU104 (10 mg/kg), (±)-STU104 (10 mg/kg), and positive drug mesalazine groups were significantly lower than those of the DSS model group, with inhibition ratios of 29.3%, 8.3%, and 8.3%, respectively (see panel B2 in FIG. 3). The (R)-STU104 group (10 mg/kg) was significantly superior to the positive drug mesalazine group (50 mg/kg). The (±)-STU104 group (10 mg/kg) was at a comparable level to the positive drug mesalazine group (50 mg/kg). On the contrary, the improvement of acute UC symptoms in mice of the (S)-STU104 group (10 mg/kg) was significantly lower than that in the mesalazine group (50 mg/kg).

### 3.3.4 Effects of Compounds (R)-STU104, (S)-STU104, and (±)-STU104 on Colon Length in DSS Induced Acute IBD Mice.

After dissection, the colon of each mouse was taken and the length of the colon was measured separately. The results showed that the colon length of the normal control group mice ranges from about 7 to 9 cm, and the intestinal mucosa was flat and smooth without any abnormalities. The DSS model group mice had occult or obvious colon bleeding, with high hardness and swelling, and ulcers and scars visible to the naked eye, and some mice even had large sections of blood soaked substances in the colon, with the colon length being shortened to 4 to 6 cm, significantly shorter than the normal control group (see panel C1 in FIG. 3). The (R)-STU104 group (10 mg/kg), (S)-STU104 group (10 mg/kg), (±)-STU104 group (10 mg/kg), and positive drug mesalazine group were all able to inhibit colon shortening to varying degrees, with recovery rates of 34.6%, 14.5%, 25.7%, and 26.4%, respectively (see panel C2 in FIG. 3). The (R)-STU104 group (10 mg/kg) was significantly superior to the (S)-STU104 group (10 mg/kg) and the positive drug mesalazine group (50 mg/kg); and the (±)-STU104 group (10 mg/kg) was at a comparable level to the positive drug mesalazine group (50 mg/kg).

### 3.3.5 Effects of Compounds (R)-STU104, (S)-STU104, and (±)-STU104 on the Pathological Condition of Colons in DSS Induced Acute IBD Mice.

The H&E staining results of colon tissues showed (see panels D1 to D3 in FIG. 3) that a large number of inflammatory cells infiltrated colon tissues of DSS induced acute IBD mice and were distributed extensively in the mucosal and submucosal layers, lymphatic tissues showed abnormal growth and had vesicles, a considerable area of tissues underwent erosion and ulceration, with a certain degree of edema. Each administrated drug and the positive control drug mesalazine showed a significant improvement in mucosal layer inflammation in mice, and the colon mucosal structures of mice were somewhat recovered, with recovery rates of 65.6%, 13.9%, 48.3%, and 38.0%, respectively. The (R)-STU104 group (10 mg/kg) was significantly superior to the positive drug mesalazine group (50 mg/kg). The (±)-STU104 group (10 mg/kg) was slightly superior to the positive drug mesalazine group (50 mg/kg). When the efficacy was superior, the administrated dose of (R)-STU104 (10 mg/kg) was only ⅕ of that of the first-line clinical control drug mesalazine (50 mg/kg). On the contrary, the recovery effect of the (S)-STU104 group (10 mg/kg) on the colon pathological condition of mice was lower than that of the positive control drug mesalazine.

3.4 Inhibitory Effects of Treatment with Compounds (R)-STU104, (S)-STU104, and (±)-STU104 on Inflammatory Factors as Experimental Endpoints of the DSS Induced Acute Ulcerative Colitis Model in C57 Mice.
3.4.1 Panels A1 to D2 in FIG. 4 Showing Effects of Compounds (R)-STU104, (S)-STU104, and (±)-STU104 and the Positive Drug Mesalazine on Protein Contents of Such Inflammatory Factors as TNF-α, IL-1β, IL-6, and IL-23 in the Serum of Mice of the DSS Induced Acute Ulcerative Colitis Model in C57 Mice.

To investigate the effects of compounds (R)-STU104, (S)-STU104, and (±)-STU104 on the secretion of such inflammatory factors as TNF-α, IL-1β, IL-6, and IL-23 by mice of the DSS induced acute ulcerative colitis model in C57 mice, we isolated serum after blood acquisition and detected the contents of such inflammatory factors as TNF-α, IL-1β, IL-6, and IL-23 in the serum of mice using ELISA. The results showed (see panels A1 to D2 in FIG. 4) that the contents of such inflammatory factors as TNF-α, IL-1β, IL-6, and IL-23 in the serum of the model group mice were significantly higher than those of the normal wild-type group. The contents of such inflammatory factors as TNF-α, IL-1β, IL-6, and IL-23 in the serum of mice in each administration group and the positive drug mesalazine group were significantly lower than those of the model group. Their inhibition ratios for four inflammatory factors are as below: 55.4%, 10.2%, 48.1%, and 42.7% for TNF-α; 70.1%, 18.1%, 68.6%, and 66.7% for IL-1β; 71.5%, 32.6%, 49.1%, and 51.1% for IL-6; and 56.7%, 18.9%, 45.2%, and 47.2% for IL-23. The (R)-STU104 group (10 mg/kg) was significantly superior to the positive drug mesalazine group (50 mg/kg). The (±)-STU104 group (10 mg/kg) was at a comparable level to the positive drug mesalazine group (50 mg/kg). When the efficacy was superior, the administrated dose of (R)-STU104 (10 mg/kg) was only ⅕ of that of the first-line clinical control drug mesalazine (50 mg/kg). On the contrary, the therapeutic effect of the (S)-STU104 group (10 mg/kg) on acute ulcerative colitis in mice was significantly lower than that of the control drug mesalazine (50 mg/kg).

3.4.2 Panels E1 to H2 in FIG. 4 Showing Effects of Treatment with Compounds (R)-STU104, (S)-STU104, and (±)-STU104 on mRNA Expressions of Such Inflammatory Factors as TNF-α, IL-1β, IL-6, and IL-23 in Colon Tissues of Mice of the DSS Induced Acute Ulcerative Colitis Model in C57 Mice.

After dissection, a segment of the colon was taken from the same part of each mouse, total RNAs were extracted from tissues, and mRNA expressions of such inflammatory factors as TNF-α, IL-1β, IL-6, and IL-23 in colon tissues of the mice were detected through qPCR. The results showed that, as shown in panels E1 to H2 of FIG. 4, the mRNA expressions of such inflammatory factors as TNF-α, IL-1β, IL-6, and IL-23 in colon tissues of the model group mice were significantly higher than those of the normal group. The mRNA expression levels of such inflammatory factors as TNF-α, IL-1β, IL-6, and IL-23 in colon tissues of the mice in both each administration group and the positive drug mesalazine group were significantly lower than those of the model group. Their inhibition ratios for four inflammatory factors are as below: 64.8%, 22.0%, 55.4%, and 41.5% for TNF-α; 68.3%, 27.7%, 67.5%, and 67.7% for IL-1β; 65.0%, 8.1%, 56.1%, and 40.2% for IL-6; and 84.8%, 10.6%, 73.1%, and 74.2% for IL-23. The (R)-STU104 group (10 mg/kg) was significantly superior to the positive drug mesalazine group (50 mg/kg); and the (±)-STU104 group (10 mg/kg) was at a comparable level to the positive drug mesalazine group (50 mg/kg). When the efficacy was superior, the administrated dose of (R)-STU104 (10 mg/kg) was only ⅕ of that of the first-line clinical control drug mesalazine (50 mg/kg). On the contrary, the therapeutic effect of the (S)-STU104 group (10 mg/kg) on acute ulcerative colitis in mice was significantly lower than that of the control drug mesalazine (50 mg/kg).

3.5 Compound (R)-STU104 for the Treatment of Chronic Ulcerative Colitis in IL-10 Whole Gene Knockout Mice in a Dose-Dependent Manner (1 mg/kg, 3 Mg/Kg, and 10 mg/kg).

Ten C57 mice (aged 10-12 weeks, around 20 g) with an identified genotype of Wildtype were taken as the normal control group. Thirty-six C57 mice (aged 10-12 weeks, around 20 g) with an identified genotype of IL-10−/− were taken and divided into the model group, positive control mesalazine group, (R)-STU104 low-dose group, (R)-STU104 medium-dose group, and (R)-STU104 high-dose group, with 10 mice in each group. C57 mice with the genotype of IL-10−/− showed a significant onset of disease around 10-12 weeks, and the experiment began after all C57 mice with the genotype of IL-10−/− had an onset of disease. The mice were administered by gavage once a day with a volume of 10 mL/kg, wherein the normal control group and DSS group were gavaged with a solvent of 0.5% CMC-Na while the other groups were separately gavaged with drugs dissolved in 0.5% CMC-Na.

### 3.5.1 Effects of Compound (R)-STU104 on Changes in Survival Rate of the Chronic Colitis Model in IL-10 Whole Gene Knockout Mice

As the experiment progressed, the model group mice had a total mortality rate of 40%, the low-dose group had a total mortality rate of 20%, and no deaths occurred in mice of the normal wild-type group, (R)-STU104 medium- and high-dose groups, and positive drug mesalazine group (see panels A1 to A2 in FIG. 5). The Kaplan-Meier survival analysis results showed that (R)-STU104 was capable of reducing the mortality rate of chronic UC mice.

### 3.5.2 Effects of Compound (R)-STU104 on Changes in Body Weight of the Chronic Colitis Model in IL-10 Whole Gene Knockout Mice.

The normal wild-type group mice were vivacious with glossy fur, normal diet and drinking, relatively fast weight gain, and no obvious abnormalities in stools or the like appearing elliptical or spindle shaped. The model group mice began to experience anal prolapse at around 8-12 weeks of age, gradually began to show a decrease in autonomous activity, dull fur, delayed response, and mental lethargy, and the mice showed a slow weight gain throughout the experiment. At the end of the experiment, the average body weight of the model group was quite significantly different from, i.e., lower than that of the normal wild-type control group (see panels B1 to B2 in FIG. 5). Compared with the model group, mice in the (R)-STU104 administration groups at different doses and the positive drug mesalazine group showed significantly lighter symptoms and faster trends in weight gain with an efficiency of 13.6%, 14.8%, 19.4%, and 17.1%, respectively. The high-dose group was superior to the positive control drug group. In addition, each administration group and the positive drug group showed increased activities and food intake, as well as significantly better mental states or the like, and showed a concentration dependent trend.

### 3.5.3 Effects of Compound (R)-STU104 on Changes in Colon Length of the Chronic Colitis Model in IL-10 Whole Gene Knockout Mice.

After dissection, the colon of each mouse was taken and the length of the colon was measured separately. The results showed that the colon length of the normal wild-type group mice was 8 to 10 cm, and the intestinal canal tissues were smooth and flat without obvious abnormalities. The intestinal wall of the model group mice was thick and congested, with obviously visible ulcers under a dissecting microscope, and the colon length was shortened to 5˜6 cm, significantly shorter than that of the normal wild-type control group. As shown (in panels C1 to C2 of FIG. 5), all dose groups of (R)-STU104 and the positive drug mesalazine group were capable of inhibiting colon shortening to varying degrees, with inhibition ratios of 17.7%, 29.4%, 41.2%, and 32.4%. The colon length in the (R)-STU104 high-dose group was significantly greater than that in the positive drug mesalazine group.

### 3.5.4 Effects of Compound (R)-STU104 on the Pathological Condition of Colons in IL-10 Whole Gene Knockout Mice.

The H&E staining results of colon tissues showed (see panels D1 to D3 in FIG. 5) that a large number of inflammatory cells infiltrated colon tissues of spontaneous IBD mice and were distributed extensively in the mucosal and submucosal layers, lymphatic tissues showed abnormal growth and had vesicles, a considerable area of tissues underwent erosion and ulceration, with a certain degree of edema. There was a significant improvement in mucosal layer inflammation in mice of each administration group, and the colon mucosal structures of mice were somewhat recovered, with recovery rates of 33.0%, 50.5%, 56.7%, and 44.3%, respectively. The high- and medium-dose groups were superior to the positive drug group. In addition, each administration group had a relatively intact epithelial structure and reduced infiltration of inflammatory cells, indicating a significant improvement compared to the model group.

3.6 Inhibitory Effects of Compound (R)-STU104 in a Dose-Dependent Manner (1 mg/kg, 3 mg/kg, and 10 mg/kg) on Inflammatory Factors as Experimental Endpoints of the Chronic Ulcerative Colitis Model in IL-10 Whole Gene Knockout Mice.

### 3.6.1 Effects of Compound (R)-STU104 on the Protein Content of Such Inflammatory Factor as TNF-α, IL-1β, IL-6, and IL-23 in the Serum of Mice of the Chronic Ulcerative Colitis Model in IL-10 Whole Gene Knockout Mice.

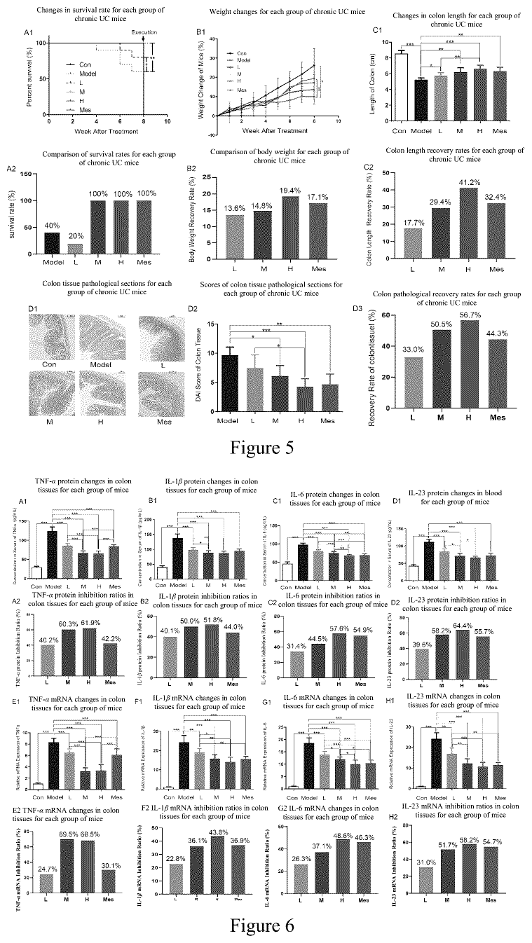

To investigate the effects of (R)-STU104 on the secretion of such inflammatory factors as TNF-α, IL-1β, IL-6, and IL-23 by spontaneous IBD mice due to IL-10 knockout, we isolated serum after blood acquisition and detected the contents of such inflammatory factors as TNF-α, IL-1β, IL-6, and IL-23 in the serum of mice using ELISA. The results showed (see panels A1 to D2 in FIG. 6) that the contents of such inflammatory factors as TNF-α, IL-1β, IL-6, and IL-23 in the serum of the model group mice were significantly higher than those of the normal wild-type group. The contents of such inflammatory factors as TNF-α, IL-1B, IL-6, and IL-23 in the serum of mice in each dose group of (R)-STU104 and the positive drug mesalazine group were significantly lower than those of the model group, and with the increase in dose, the contents of these inflammatory factors in the serum of spontaneous IBD mice showed a decreasing trend. Their inhibition ratios for four inflammatory factors are as below: 40.2%, 60.3%, 61.9%, and 42.2% for TNF-α; 40.1%, 50.0%, 51.8%, and 44.0% for IL-1β; 34.4%, 44.5%, 57.6%, and 54.9% for IL-6; and 39.6%, 58.2%, 64.4%, and 55.7% for IL-23. Overall, the high- and medium-dose groups were superior to the positive drug control group.

3.6.2 Effects of Compound (R)-STU104 on mRNA Expressions of Such Inflammatory Factor as TNF-α, IL-1β, IL-6, and IL-23 in Colon Tissues of Mice of the Chronic Ulcerative Colitis Model in IL-10 Whole Gene Knockout Mice.

After dissection, a segment of the colon was taken from the same part of each mouse, total RNAs were extracted from tissues, and mRNA expressions of such inflammatory factors as TNF-α, IL-1β, IL-6, and IL-23 in colon tissues of the mice were detected through qPCR. The results showed that, as shown in panels E1 to H2 of FIG. 6, the mRNA expressions of such inflammatory factors as TNF-α, IL-1β, IL-6, and IL-23 in colon tissues of the model group mice were significantly higher than those of the normal group. The mRNA expression levels of such inflammatory factors as TNF-α, IL-1β, IL-6, and IL-23 in colon tissues of the mice in both each dose group of (R)-STU104 and the positive drug mesalazine group were significantly lower than those of the model group, and showed a significant dose dependence. Their inhibition ratios for four inflammatory factors are as below: 24.7%, 69.5%, 68.5%, and 30.1% for TNF-α; 22.8%, 36.1%, 43.8%, and 36.9% for IL-1β; 26.3%, 37.1%, 48.6%, and 46.3% for IL-6; and 31.0%, 51.7%, 58.2%, and 54.7% for IL-23. Overall, the high- and medium-dose groups were superior to the positive drug control group.

The above provides a detailed description of preferred specific embodiments of the present invention. It should be understood that those of ordinary skill in the art can make many modifications and changes based on the concept of the present invention without creative labor. Therefore, all technical solutions available to a person skilled in the art based on the concept of the present invention and existing technology through logical analysis, reasoning, or limited experiments should fall within the scope of protection determined by the claims.

1. A small molecule TNF-α inhibitor or a pharmaceutically acceptable salt thereof, wherein its structure is represented by formula I:

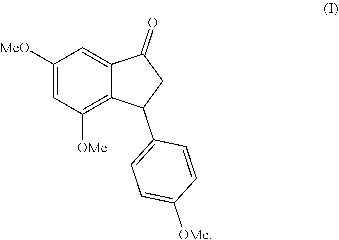 [SMILES: COc1ccc(C2CC(=O)c3cc(OC)cc(OC)c32)cc1]

2. The small molecule TNF-α inhibitor or the pharmaceutically acceptable salt thereof as recited in claim 1, wherein said small molecule TNF-α inhibitor or the pharmaceutically acceptable salt thereof is racemic (±)-STU104 or (R)-STU104, i.e., 4,6-dimethoxy-3R-(4-methoxyphenyl)-2,3-dihydro-1H-indanone with a structure represented by formula II or III:

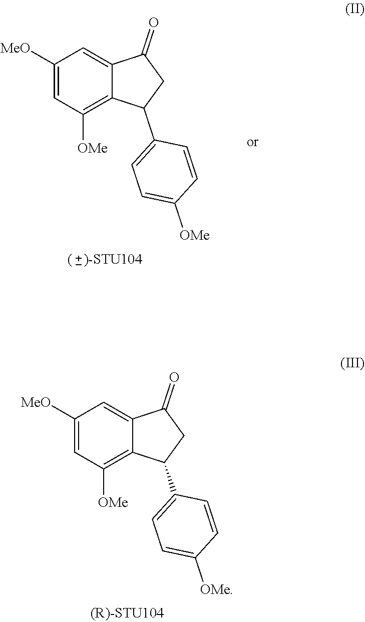

3. A method for preparation of the small molecule TNF-α inhibitor (R)-STU104 of formula III according to claim 2, wherein it is prepared according to the following route:

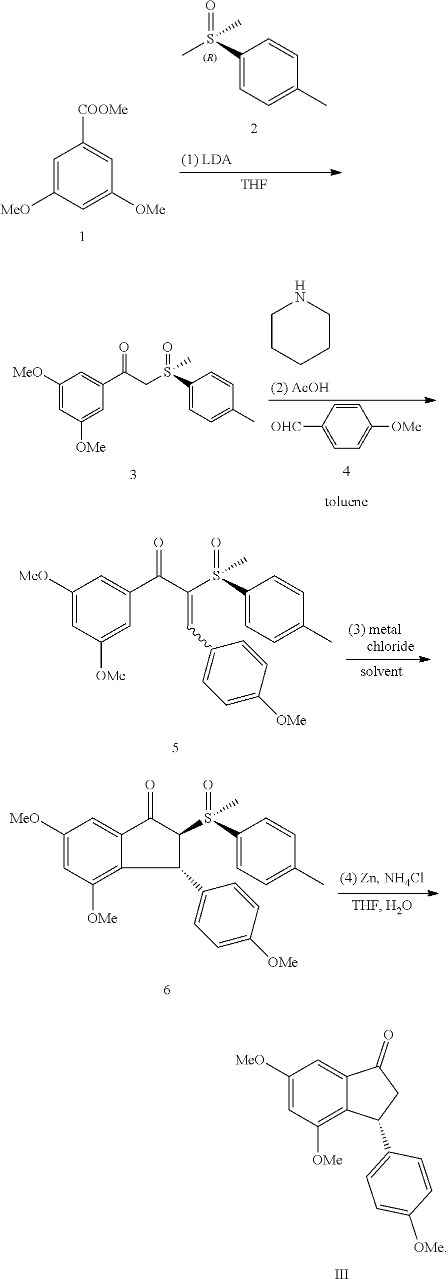

4. The method for preparation of the small molecule TNF-α inhibitor (R)-STU104 of formula III as recited in claim 3, wherein in Step (1), a synthetic reaction of Compound 3 from Compounds 1 and 2 is controlled at a temperature of −80 to −60° C.

5. The method for preparation of the small molecule TNF-α inhibitor (R)-STU104 of formula III as recited in claim 3, wherein in Step (2), a synthetic reaction of Compound 5 from Compounds 3 and 4 is controlled at a temperature of 90 to 120° C.

6. The method for preparation of the small molecule TNF-α inhibitor (R)-STU104 of formula III as recited in claim 3, wherein in Step (3), a synthetic reaction of Compound 6 from Compound 5 is controlled at a temperature of 30 to 50° C.

7. The method for preparation of the small molecule TNF-α inhibitor (R)-STU104 of formula III as recited in claim 3, wherein in Step (3), a metal chloride used in the synthetic reaction of Compound 6 from Compound 5 is AlCl₃ or ZnCl₂.

8. The method for preparation of the small molecule TNF-α inhibitor (R)-STU104 of formula III as recited in claim 3, wherein a solvent used in the synthetic reaction of Compound 6 from Compound 5 is toluene, dichloromethane, or 1,2-dichloroethane.

9. Use of the small molecule TNF-α inhibitor or the pharmaceutically acceptable salt thereof according to claim 1 in preparation of medicaments for treating autoimmune inflammatory diseases.

10. The use as recited in claim 9, wherein said autoimmune inflammatory diseases are caused by overexpression of TNF-α or overexpression of mRNAs thereof.

11. The use as recited in claim 9, wherein said use is to have an effect of inhibiting or reducing TNF-α release; furthermore, said use is to reduce a phosphorylation level of MKK3 by regulating TAK1 within inflammatory cells, and further mediate inhibition or reduction of phosphorylation levels of proteins including but not limited to p38, MnK1, MK2, and/or elF4E in a downstream signaling pathway of MKK3 to achieve the effect of inhibiting or reducing the TNF-α release.

12. The use as recited in claim 9, wherein said autoimmune inflammatory diseases include but are not limited to ulcerative bowel diseases, rheumatoid arthritis, osteoarthritis, psoriasis, alopecia areata, Sjogren's syndrome, lupus erythematosus or dermatomyositis.

13. The use as recited in claim 12, wherein said ulcerative bowel diseases include but are not limited to acute ulcerative colitis, chronic ulcerative colitis, or Crohn's disease.

14. The use as recited in claim 13, wherein treatment of said acute ulcerative colitis or said chronic ulcerative colitis is to have an effect of inhibiting overexpression of at least one of optional inflammatory factors TNF-α, IL-1β, IL-6, and IL-23 or overexpression of mRNAs thereof.

15. The use as recited in claim 13, wherein said treatment of said chronic ulcerative colitis is dose-dependent.

16. The use as recited in claim 13, wherein treatment of said acute ulcerative colitis, said chronic ulcerative colitis, or said Crohn's disease includes but is not limited to improvement in symptoms and reduction in mortality rates; further, said improvement in symptoms includes but is not limited to inhibiting weight loss, reducing hematochezia, and inhibiting colon shortening, ulcers, scars, and/or edema.
